(19) United States
(12) Patent Application Publication
(10) Pub. No.: US 2025/0040925 A1
(43) Pub. Date: Feb. 6, 2025

(54) SUTURE STRUCTURE THAT ENHANCES LIFTING EFFECT

(71) Applicant: DIAMOND BIOTECHNOLOGY CO., LTD., New Taipei City (TW)

(72) Inventor: CHIA HSIEN HSIEH, New Taipei City (TW)

(21) Appl. No.: 18/646,789

(22) Filed: Apr. 26, 2024

(30) Foreign Application Priority Data
Aug. 4, 2023 (TW) 112129357

(51) Int. Cl. A61B 17/06; A61F 2/00

(52) U.S. Cl. CPC A61B 17/06166; A61F 2/0059; A61B 2017/06176; A61F 2220/0016

(57) ABSTRACT

Disclosed is a suture structure that enhances lifting effect, including: a thread having a thread diameter; a plurality of barbs arranged on a surface of the thread and including at least one planar surface; and a plurality of grooves arranged adjacent to the barbs at one side. The barbs have a barb height and a barb width, and the grooves have a groove length and a groove depth. The barb height is between 0.2-0.35 times of the thread diameter; the barb width is between 0.35-0.5 times of the thread diameter; the groove length is between 0.5-0.8 times of the thread diameter; and the groove depth is between 0.3-0.5 times of the thread diameter. The barbs are arranged on the surface of the thread, and the groove depth is 0.33 times of the thread diameter, so that the structural strength is relatively great.

### BACKGROUND OF THE INVENTION

### (a) Technical Field of the Invention

The present invention relates to a suture structure, and more particularly to a suture structure that enhances a lifting effect.

### (b) Description of the Prior Art

Thread lift (skin tightening) is a surgery procedure that involves a surgery suture or thread of a special specification and implants the thread under a skin in a manner of being introduced with a needle tube, where the thread includes barbs that fix the thread on fascia for concentration and lifting so as to increase an upward force on the skin, making the skin stretched and flexible, and the characteristics of the thread as being absorbable may simulate collagen to proliferate so as to induce a progressively increasing effect to have the skin restore tightening and flexible and also lift up the loose muscles and skins.

As provided in Taiwan Utility Model, M580945, which discloses a “suture for plastic surgery”, the suture includes a thread and multiple sharp protrusions. Dual-blade cutting is adopted to carry out cutting on a surface of the thread in identical directions or in opposite directions in one spiraling angle so as to make multiple cut notches on the surface of the thread, and would be one-to-one corresponding to the sharp protrusions that are for pull of skin after the cutting for each cut notch is done. Further, Taiwan Patent, 1785810, discloses an “introduction needle”, and as shown in FIG. 6 of the 1785810 patent, a suture is cut to a desired length, and the suture is placed in the interior of the introduction needle and reaching out of an opening of the introduction needle. To use in a surgery operation, the operator first uses a sharp object to puncture the skin of a surgery subject and penetrates the introduction needle into the inside of the skin of the surgery subject to reach a predetermined location. Then, an extra portion of the suture is cut off and the introduction needle is slowly withdrawn to allow a desired portion of the suture to be left inside the skin of the surgery subject.

However, as shown in FIGS. 1 and 2 of Utility Model M580945, the suture of the Utility Model is made by applying dual blade cutting to make cutting on the surface of the thread to provide multiple cut notches and multiple sharp protrusion on the thread. The cut notches have a relatively small notch length and are set in an inclined manner, so that cut notches do not provide a sufficient space for receiving and holding the human body tissues, and consequently, it only relies on the sharp protrusions to grip the human body tissues. However, the sharp protrusion may stab into the human body tissues, increasing perception of pains. Further, as seen in FIGS. 1 and 2 of Utility Model M580945, to allow the sharp protrusions to protrude from the surface of the thread and warp up, the cutting depth reaches almost one half of the thread diameter.

This causes a significant damage to the structure of the thread, making the structural strength relatively weak, resulting in easy breaking.

### SUMMARY OF THE INVENTION

To achieve the objective of the present invention, the present invention provides a suture structure that enhances lifting effect, comprising: a thread, which has a thread diameter; a plurality of barbs, which are arranged on a surface of the thread, the barbs having at least one planar surface; and a plurality of grooves, which are arranged adjacent to the barbs at one side, wherein the barbs have a barb height and a barb width, and the grooves have a groove length and a groove depth, the barb height being between 0.2-0.35 times of the thread diameter, the barb width being between 0.35-0.5 times of the thread diameter, the groove length being between 0.5-0.8 times of the thread diameter, the groove depth being between 0.3-0.5 times of the thread diameter.

In the above, the barb height is 0.27 times of the thread diameter.

In the above, the barb width is 0.46 times of the thread diameter.

In the above, the groove length is 0.55 times of the thread diameter.

In the above, the groove depth is 0.33 times of the thread diameter.

In the above, the plurality of barbs are arranged on the thread in a straight line manner or a spiral manner.

In the above, the plurality of grooves are arranged on the thread in a straight line manner or a spiral manner.

In the above, the plurality of barbs are arranged on the thread in an equidistant manner or a non-equidistant manner.

In the above, the plurality of grooves are arranged on the thread in an equidistant manner or a non-equidistant manner.

In the above, the grooves are a planar groove or a circular arc groove.

Compared to the prior art technology, the present invention shows the following advantages for development:

(1) Since the barbs are provided on the surface of the thread and the groove depth is 0.33 times of the thread diameter, the structural strength is relatively great and breaking does not easily occur. Oppositely, in the prior art, the barbs are formed by cutting into the surface of the thread, and to make the barbs warping up, the cutting depth is almost one half of the thread diameter, and thus, the structure of the thread is damaged relatively significant and the structural strength is relatively low, making it easy to break.

(2) The barbs are provided with at least one planar surface, so that the barbs are not made in a sharp form, reducing the risk of hurt by stabbing. Oppositely, in the prior art, the barbs are made in the form of a sharpened form, which may easily stab into and hurt human body tissues.

(3) The groove length is between 0.5 to 0.8 times of the thread diameter, and this increases the surface area of the thread and the grooves are provided with a sufficient space to receive and hold the human body tissues to help improve secureness.

Oppositely, in the prior art, the surface of the thread is formed with multiple inclined cut notches and the length of the cut notches is relatively small, so that there is no sufficient space for receiving and holding the human body tissues, and thus, it only relies on the sharp protrusions to grip the human body tissue, and further, the grooves are prevented from being made excessively large, and this makes the grooves losing the capability of receiving and holding the tissues and reducing the strength of the thread, equivalently making the entire thread very thin.

(4) The barb width is set between 0.35 to 0.5 times of the thread diameter, and this provides the barbs with a sufficient thickness and the time for degradation is extended, and also helps prevent the risk of losing the configuration of barb, which results in loss of the lifting effect, caused by the thickness being made large.

(5) The groove depth is set between 0.3 to 0.5 times of the thread diameter, and this provides the grooves with a sufficient depth to allow the human body tissues to be received and held in the grooves to thereby more securely hold the human body tissues.

(6) The barb height is set between 0.2 to 0.35 times of the thread diameter, and this provides the barbs with a sufficient height to grip the human body tissue and also helps prevent the risk that the barbs are folded backward due to an excessive height of the barbs, to loss the lifting effect.

### BRIEF DESCRIPTION OF THE DRAWINGS

FIG. 1 is a schematic view showing a suture structure that enhances lifting effect according to the present invention.

FIG. 2 is a schematic view showing barbs arranged on a thread in a spiral arrangement according to the present invention.

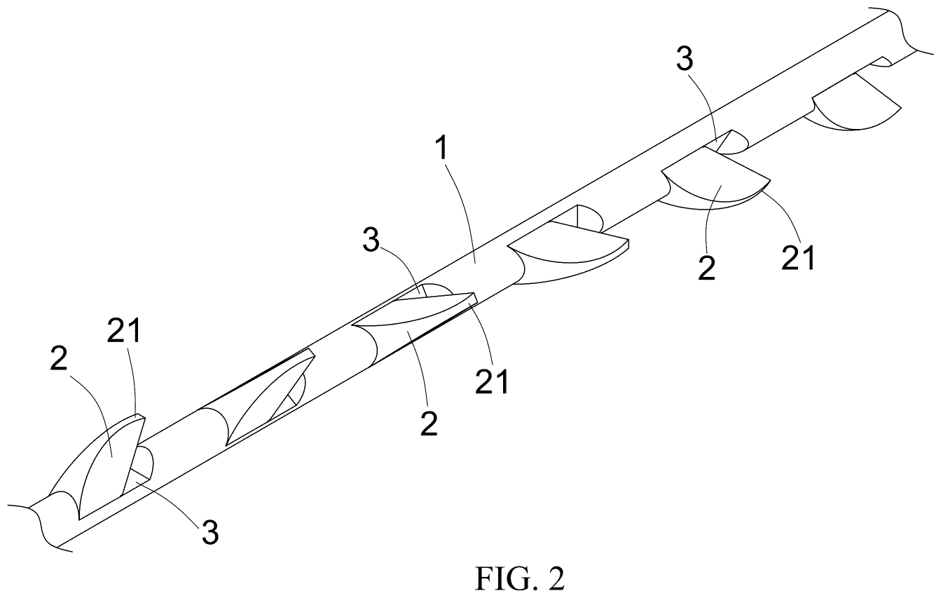

FIG. 3 is a schematic view showing barbs arranged on a thread in a non-equidistant arrangement according to the present invention.

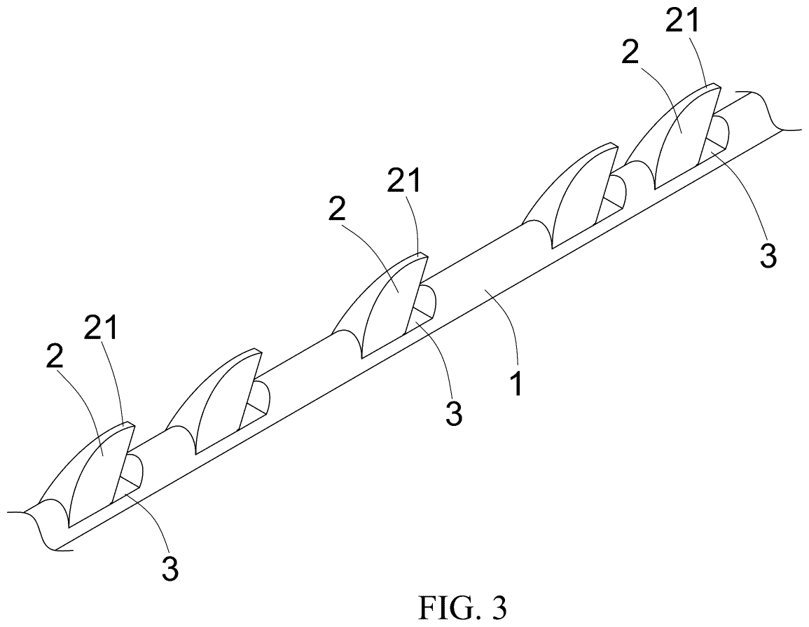

FIG. 4 is a top plan view showing the suture that enhances lifting effect according to the present invention.

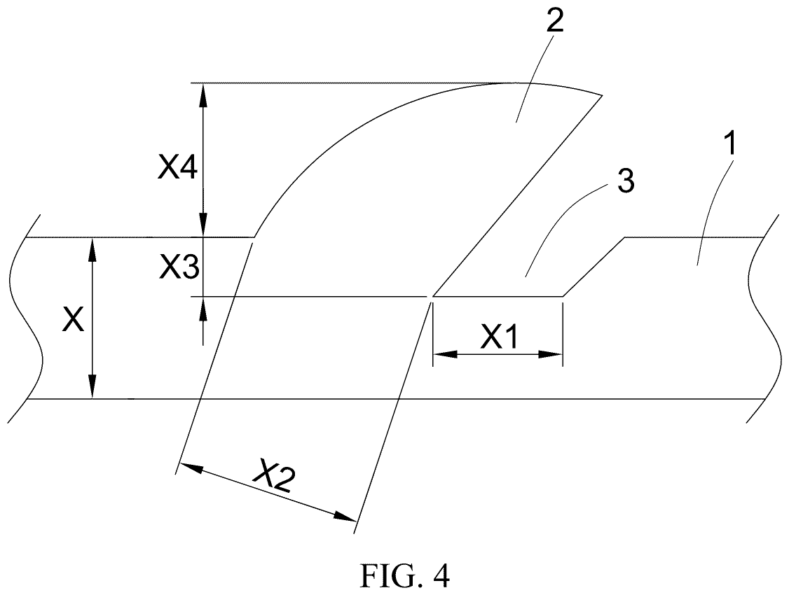

FIG. 5 is a photographed picture showing a suture clamped in a tension test machine.

FIG. 6 is a photographed picture showing a pork skin tissue clamped in a tension test machine.

FIG. 7 is a photographed picture showing a suture penetrating through a pork skin tissue.

### DETAILED DESCRIPTION OF THE PREFERRED EMBODIMENTS

For easy and clear understanding of other features and advantages of the present invention, and the effectiveness achieved thereby, a detailed description of the present invention will be provided below, with reference to the attached drawings.

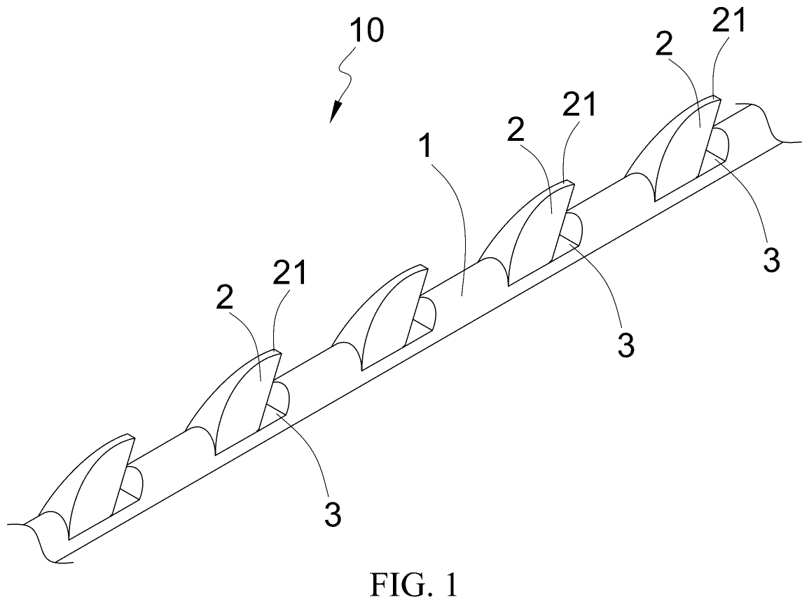

Referring to FIG. 1, the present invention provides a suture structure that enhances lifting effect, comprising: a thread 1, a plurality of barbs 2, and a plurality of grooves 3.

The thread 1 has a thread diameter X. The thread 1 can be produced with production procedures that are commercially known, such as cutting, molding, and injection. The thread 1 can be coated with a material that helps repair and restore skins, such as collagen. Classified by material, the thread 1 can be divided as absorbable and nonabsorbable. The absorbable materials include, for example, plain catgut, chromic gut, Dexon, Vicryl, polycaprolactone (PCL), polypropylene carbonate (PPC), polytrimethylene carbonate, polydioxanone (PDO), PGA, PLGA, LA, and polydioxanone (PDS), and the nonabsorbable materials include, for example, silk, cotton, nylon, polyester, polyethylene, polypropylene, polytetrafluoroethylene, stainless steel, and polybutester. In the instant embodiment, the material of the thread 1 is PDO or polycaprolactone (PCL). Similar to other sutures, the PDO thread can be classified for three types, which are smooth thread, spiral thread, and sawtooth thread, and the sawtooth thread is further divided as plural types, such as unidirectional sawtooth, bidirectional sawtooth, and staggered sawtooth. The PDO thread and the PCL thread are primarily concerned with service life and tensile strength, and the PCL thread is durable than the PDO thread and exhibits a higher tensile strength.

The plurality of barbs 2 are arranged on a surface of the thread 1, and the barbs 2 each include at least one planar surface 21. The plurality of barbs 2 are arranged on the thread 1 in a straight line arrangement or a spiral arrangement. The plurality of barbs 2 are arranged on the thread 1 in an equidistant manner or a non-equidistant manner.

The plurality of grooves 3 are arranged adjacent to the barbs 2 at one side thereof, wherein the barbs 2 have a barb height X4 and a barb width X2, and the grooves 3 have a groove length X1 and a groove depth X3. The barb height X4 is between 0.2 to 0.35 times of the thread diameter X, and the barb width X2 is between 0.35 to 0.5 times of the thread diameter X. The groove length X1 is between 0.5 to 0.8 times of the thread diameter X, and the groove depth X3 is between 0.3 to 0.5 times of the thread diameter X. The grooves 3 can be a flat groove or a circular arc groove. Tests made on the barb height X4, the barb width X2, the groove length X1, and the groove depth X3 of the suture structure 10 indicate the preferable values thereof are as follows: The barb height X4 is 0.27 times of the thread diameter X; the barb width X2 is 0.46 times of the thread diameter X; the groove length X1 is 0.55 times of the thread diameter X; and the groove depth X3 is 0.33 times of the thread diameter X.

The suture structure 10 of the present invention have been subjected to tensile tests and degradation tests, of which the processes and data are as follows:

### Tensile Test

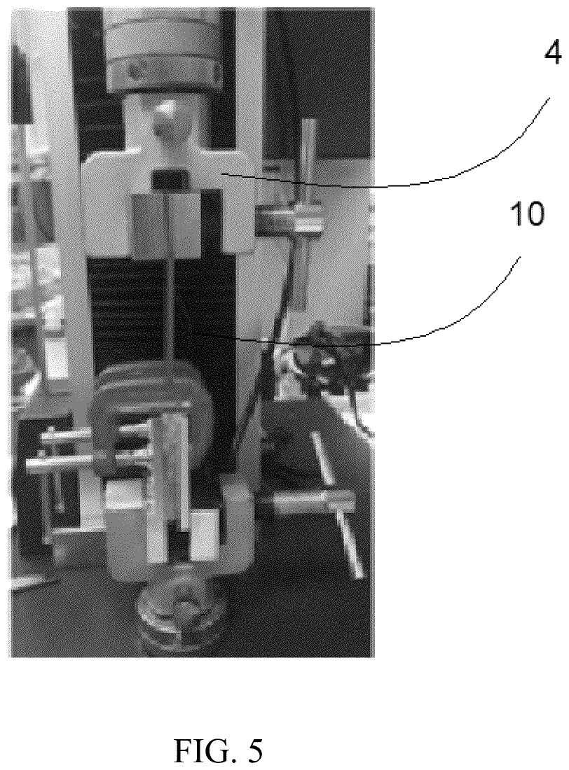

Principle: The experimental principle of the method is that a test sample is cut into strips of the same size with a specific cutting machine, and the sample so cut is clamped in a tension test machine 4 (as shown in FIG. 5) and is subject to elongating at a constant speed until the sample breaks, indicating the force that the sample can bear. Methodology: operating the tension test machine 4 (as shown in FIG. 5), clamping the suture structure 10 in the top and bottom clamps of a material test machine, avoiding sliding of the suture structure 10, setting the elongating speed to 250 mm/min, fixing points of the clamps being necessarily spaced from two ends of the suture structure 10 larger than 13 mm, two ends of the clamps being between 125-200 mm, where during elongating the suture structure 10 in the tension test machine 4, if the breaking point is at the site of the clamps, the test must be re-performed.

### Data Result of Tensile Test

A PDO suture of USP2 is used to carry out a cutting operation and various conditions are applied to test the thread property of the suture.

(Only following experiment groups 2, 3, 4 reach acceptable level.)

TABLE 1

| | | | | | | Experiment Item | | |
|---|---|---|---|---|---|---|---|---|
| | | | | | | Tensile | | Barb |
| | | | | | | Strength | Lifting | Degradation |
| | | | | | | (kgf) | Strength (N) | Time (days) |
| | | | | | | (required | (required to | (required to |
| | | | | | | to be | be >9.05 N) | be >88.6 days) |
| | | | | | | >3.90 kgf) | Stronger | Longer |
| Experiment | Parameter (mm) | | | | | Satisfying | than known | than known |
| Group | X | X1 | X2 | X3 | X4 | USP | barbed thread | barbed thread |
| 1 | 0.6 | 0.5 | 0.35 | 0.2 | 0.25 | 3.10 | 4.45 | 134.5 days |
| | | (NA) | (NA) | | (NA) | (NA) | (NA, showing | |
| | | | | | | | thread back | |
| | | | | | | | folding) | |
| 2 | 0.6 | 0.48 | 0.3 | 0.3 | 0.21 | 5.66 | 12.95 | 129.3 days |
| 3 | 0.6 | 0.3 | 0.21 | 0.18 | 0.12 | 6.12 | 11.08 | 95.47 days |
| 4 | 0.6 | 0.33 | 0.28 | 0.20 | 0.16 | 7.62 | 13.44 | 130.1 days |
| 5 | 0.6 | 0.3 | 0.21 | 0.15 | 0.13 | 5.98 | 8.77 | 112.5 days |
| | | | | (NA) | | | (NA) | |
| 6 | 0.6 | 0.3 | 0.28 | 0.34 | 0.22 | 2.76 | 8.41 | 110.4 days |
| | | | | (NA) | (NA) | (NA) | (NA) | |

NA indicating “not acceptable” as not meeting requirements

### Lifting Test

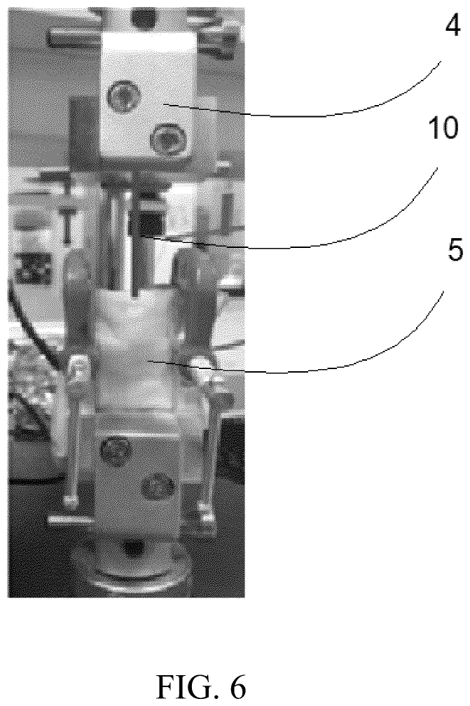

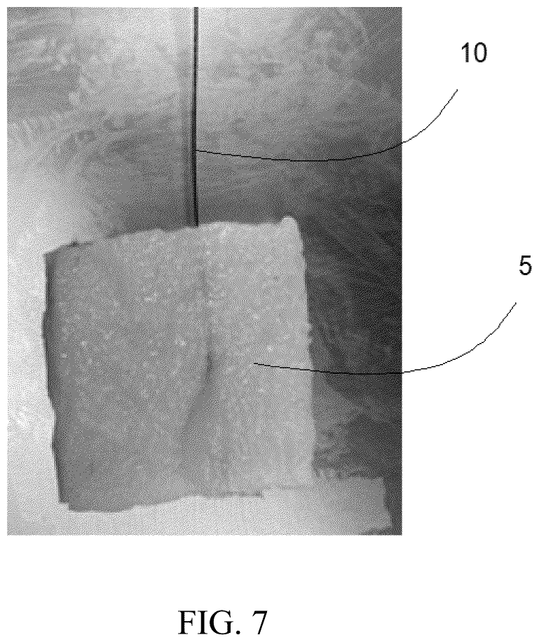

On the day of test, a fresh pork skin tissue 5 was purchased and tissue samples are prepared with surgical knives by cutting the port skin tissue into a size of approximately 5 cm×5 cm×1 cm, and the suture structure 10 is made penetrating through the pork skin tissue 5 (as shown in FIG. 7), where an implanted length must be 50±2 millimeters, and a penetrating region must be one carrying the barbs, and after implantation, an extra portion of the suture structure 10 is removed, and the pork skin tissue 5 is set as being clamped in the tension test machine 4 (as shown in FIG. 6), with the distance between the clamps being 100 mm, and moving speed being 10 mm/min.

### Data Result of Lifting Test

A PDO suture of USP2 is used to carry out a die pressing operation and various conditions are applied to test the thread property of the suture.

(Only following experiment groups 2, 3, 4 reach acceptable level.)

TABLE 2

| | | | | | | Experiment Item | | |
|---|---|---|---|---|---|---|---|---|
| | | | | | | Tensile | | Barb |
| | | | | | | Strength | Lifting | Degradation |
| | | | | | | (kgf) | Strength (N) | Time (days) |
| | | | | | | (required | (required to | (required to be |
| | | | | | | to be | be >11.6 N) | >220.4 days) |
| | | | | | | >3.90 kgf) | Stronger | Longer |
| Experiment | Parameter (mm) | | | | | Satisfying | than known | than known |
| Group | X | X1 | X2 | X3 | X4 | USP | barbed thread | barbed thread |
| 2 | 0.6 | 0.51 | 0.32 | 0.25 | 0.22 | 2.67 | 3.87 | 288.1 days |
| | | (NA) | (NA) | | (NA) | (NA) | (NA, showing | |
| | | | | | | | thread back | |
| | | | | | | | folding) | |
| 3 | 0.6 | 0.49 | 0.31 | 0.3 | 0.21 | 6.18 | 14.31 | 264.7 days |
| 4 | 0.6 | 0.3 | 0.21 | 0.19 | 0.12 | 6.77 | 13.56 | 235.9 days |
| 5 | 0.6 | 0.33 | 0.28 | 0.20 | 0.16 | 8.62 | 15.41 | 279.4 days |
| 6 | 0.6 | 0.3 | 0.21 | 0.15 | 0.14 | 6.11 | 6.21 | 262.8 days |
| | | | | (NA) | | | (NA) | |
| 7 | 0.6 | 0.3 | 0.21 | 0.32 | 0.24 | 3.20 | 10.55 | 244.8 days |
| | | | | (NA) | (NA) | (NA) | (NA, showing | |
| | | | | | | | thread back | |
| | | | | | | | folding) | |

NA indicating “not acceptable” as not meeting requirements

Further, during a degradation course of the suture structure 10, the tensile strength and the elongation rate are first reduced, and in the present invention, since the tensile strength is increased, the degradation speed is relatively reduced to thereby extend the degradation time.

Compared to the prior art technology, the present invention shows the following advantages for development:

(1) Since the barbs are provided on the surface of the thread and the groove depth is 0.33 times of the thread diameter, the structural strength is relatively great and breaking does not easily occur. Oppositely, in the prior art, the barbs are formed by cutting into the surface of the thread, and to make the barbs warping up, the cutting depth is almost one half of the thread diameter, and thus, the structure of the thread is damaged relatively significant and the structural strength is relatively low, making it easy to break.

(2) The barbs are provided with at least one planar surface, so that the barbs are not made in a sharp form, reducing the risk of hurt by stabbing. Oppositely, in the prior art, the barbs are made in the form of a sharpened form, which may easily stab into and hurt human body tissues.

(3) The groove length is between 0.5 to 0.8 times of the thread diameter, and this increases the surface area of the thread and the grooves are provided with a sufficient space to receive and hold the human body tissues to help improve secureness. Oppositely, in the prior art, the surface of the thread is formed with multiple inclined cut notches and the length of the cut notches is relatively small, so that there is no sufficient space for receiving and holding the human body tissues, and thus, it only relies on the sharp protrusions to grip the human body tissue, and further, the grooves are prevented from being made excessively large, and this makes the grooves losing the capability of receiving and holding the tissues and reducing the strength of the thread, equivalently making the entire thread very thin.

(4) The barb width is set between 0.35 to 0.5 times of the thread diameter, and this provides the barbs with a sufficient thickness and the time for degradation is extended, and also helps prevent the risk of losing the configuration of barb, which results in loss of the lifting effect, caused by the thickness being made large.

(5) The groove depth is set between 0.3 to 0.5 times of the thread diameter, and this provides the grooves with a sufficient depth to allow the human body tissues to be received and held in the grooves to thereby more securely hold the human body tissues.

(6) The barb height is set between 0.2 to 0.35 times of the thread diameter, and this provides the barbs with a sufficient height to grip the human body tissue and also helps prevent the risk that the barbs are folded backward due to an excessive height of the barbs, to loss the lifting effect.

I claim:

1. A suture structure that enhances lifting effect, comprising:
a thread, which has a thread diameter;
a plurality of barbs, which are arranged on a surface of the thread, the barbs having at least one planar surface; and
a plurality of grooves, which are arranged adjacent to the barbs at one side, wherein the barbs have a barb height and a barb width, and the grooves have a groove length and a groove depth, the barb height being between 0.2-0.35 times of the thread diameter, the barb width being between 0.35-0.5 times of the thread diameter, the groove length being between 0.5-0.8 times of the thread diameter, the groove depth being between 0.3-0.5 times of the thread diameter.

2. The suture structure that enhances lifting effect according to claim 1, wherein the barb height is 0.27 times of the thread diameter.

3. The suture structure that enhances lifting effect according to claim 1, wherein the barb width is 0.46 times of the thread diameter.

4. The suture structure that enhances lifting effect according to claim 1, wherein the groove length is 0.55 times of the thread diameter.

5. The suture structure that enhances lifting effect according to claim 1, wherein the groove depth is 0.33 times of the thread diameter.

6. The suture structure that enhances lifting effect according to claim 1, wherein the plurality of barbs are arranged on the thread in a straight line manner or a spiral manner.

7. The suture structure that enhances lifting effect according to claim 1, wherein the plurality of grooves are arranged on the thread in a straight line manner or a spiral manner.

8. The suture structure that enhances lifting effect according to claim 1, wherein the plurality of barbs are arranged on the thread in an equidistant manner or a non-equidistant manner.

9. The suture structure that enhances lifting effect according to claim 1, wherein the plurality of grooves are arranged on the thread in an equidistant manner or a non-equidistant manner.

10. The suture structure that enhances lifting effect according to claim 1, wherein the grooves are a planar groove or a circular arc groove.
